(19) United States
(12) Patent Application Publication
(10) Pub. No.: US 2021/0024433 A1
(43) Pub. Date: Jan. 28, 2021

(54) SOLID COMPOSITION CONTAINING OXIDIZED GLUTATHIONE AND METHOD FOR PRODUCING SOLID COMPOSITION

(71) Applicant: KANEKA CORPORATION, Osaka (JP)

(72) Inventors: Takayuki Asada, Hyogo (JP); Yoshiyuki Nakano, Hyogo (JP); Toyoaki Watanabe, Hyogo (JP); Ken Uekita, Hyogo (JP)

(73) Assignee: KANEKA CORPORATION, Osaka (JP)

(21) Appl. No.: 17/039,471

(22) Filed: Sep. 30, 2020

Related U.S. Application Data
(63) Continuation of application No. PCT/JP2019/013942, filed on Mar. 29, 2019.

(30) Foreign Application Priority Data
Mar. 30, 2018 (JP) 2018-066497

(51) Int. Cl. C05G 5/10

(52) U.S. Cl. CPC C05G 5/10

(57) ABSTRACT

One or more embodiments of the present invention provide an oxidized glutathione-containing solid composition which can be produced by a simple method and has low deliquescence and is easy to handle. One or more embodiments of the present invention relate to a solid composition comprising an amorphous oxidized glutathione and a water-soluble cellulose derivative. One or more embodiments of the present invention also relate to a method for producing the solid composition comprising an amorphous oxidized glutathione and a water-soluble cellulose derivative, comprising a step of drying a solution comprising oxidized glutathione and a water-soluble cellulose derivative dissolved in an aqueous solvent.

### TECHNICAL FIELD

One or more embodiments of the present invention relate to a solid composition comprising oxidized glutathione with suppressed deliquescence and a method for producing the same.

### BACKGROUND

Oxidized glutathione (GSSG), as well as reduced glutathione (GSH), is useful in many fields including health food products, pharmaceutical products, cosmetic products, and fertilizers.

Oxidized glutathione (GSSG) is a molecule obtained when 2 molecules of reduced glutathione (GSH) are oxidized and form a disulfide bond. Reduced glutathione is composed of a tripeptide consisting of glutamic acid, cysteine and glycine.

A known method for producing oxidized glutathione is, for example, a method comprising preparing an aqueous solution of reduced glutathione and then oxidizing the obtained aqueous solution to produce an aqueous solution of oxidized glutathione or alternatively a method comprising preparing a yeast solution by fermentation and then oxidizing the obtained yeast solution to produce an aqueous solution of oxidized glutathione (Patent Literature 1).

Patent Literature 1 describes a method for producing a yeast extract powder containing oxidized glutathione-containing comprising oxidizing a yeast solution to prepare an oxidized glutathione-containing yeast extract solution, mixing one or two or more selected from excipients consisting of dextrin, gelatin, casein sodium, lactalbumin and albumen with the yeast extract solution, and then drying the mixture.

Patent Literature 2 aims at providing a solid oxidized glutathione which is low in deliquescency and is easily handled and at the same time, is highly water-soluble. Patent Literature 2 discloses a method for producing a solid oxidized glutathione salt, comprising heating oxidized glutathione to a temperature of 30° C. or more while the oxidized glutathione is contacted with an aqueous medium comprising water and/or water-soluble medium in the presence of a substance capable of generating at least one cation selected from an ammonium cation, a calcium cation and a magnesium cation to generate a salt of the oxidized glutathione and the cation as a solid.

Patent Literature 3 discloses a non-crystalline amorphous oxidized glutathione produced by drying a crystal of oxidized glutathione hexahydrate at a temperature of 40 to 90° C. Patent Literature 3 discloses that this amorphous oxidized glutathione is an oxidized glutathione powder that has excellent stability and is handled industrially with ease,

Patent Literature 4 describes a composition comprising oxidized glutathione and sodium carboxymethyl cellulose or a salt thereof, as a controlled-release fertilizer comprising oxidized glutathione as an active component.
Patent Literature 1: JP Patent Publication (Kokai) No. 5-146279
Patent Literature 2: International Publication No. WO2013/002317
Patent Literature 3: International Publication No. WO2011/132725
Patent Literature 4: International Publication No. WO2016/129512

### SUMMARY

The oxidized glutathione-containing yeast extract powder described in Patent Literature 1 poses the problem of having high deliquescence and being difficult to handle.

The oxidized glutathione salt crystal described in Patent Literature 2 has low deliquescence and excellent handle-ability. However, the production of this oxidized glutathione salt crystal needs the generation of crystals by crystallization thus limiting industrial utilization from the viewpoint of time and cost.

The method for producing a non-crystalline amorphous oxidized glutathione described in Patent Literature 3 also needs crystals of oxidized glutathione hexahydrate as a raw material thus limiting industrial utilization from the viewpoint of time and cost as does the method described in Patent Literature 2.

Thus, one or more embodiments of the present invention provide, to solve the problem, an oxidized glutathione-containing solid composition which can be produced by a simple method and has low deliquescence and is easy to handle.

One or more embodiments of the present invention encompass the following inventions.
(1) A method for producing a solid composition comprising an amorphous oxidized glutathione and a water-soluble cellulose derivative, the method comprising
a step of drying a solution comprising oxidized glutathione and a water-soluble cellulose derivative dissolved in an aqueous solvent.
(2) The method according to (1), wherein the step comprises drying the solution by spray drying, freeze drying or drum drying.
(3) The method according to (1) or (2), wherein the solution comprises 1 to 50 parts by mass of the water-soluble cellulose derivative per 100 parts by mass of the oxidized glutathione.
(4) The method according to any of (1) to (3), wherein the solution comprises 20 mass % or more of the oxidized glutathione based on the total amount of components other than the solvent.
(5) The method according to any of (1) to (4), wherein the solution comprises 70 mass % or more, in total, of the oxidized glutathione and the water-soluble cellulose derivative based on the total amount of components other than the solvent.
(6) A solid composition comprising an amorphous oxidized glutathione and a water-soluble cellulose derivative.
(7) The solid composition according to (6), comprising 1 to 50 parts by mass of the water-soluble cellulose derivative per 100 parts by mass of the amorphous oxidized glutathione.
(8) The solid composition according to (6) or (7), comprising 20 mass % or more of the amorphous oxidized glutathione based on the total amount of the solid composition.
(9) The solid composition according to any of (6) to (8), produced by a method comprising a step of drying a solution comprising oxidized glutathione and a water-soluble cellulose derivative dissolved in an aqueous solvent.
(10) The solid composition according to (9), wherein the step comprises drying the solution by spray drying, freeze drying or drum drying.
(11) The solid composition according to any of (6) to (10), comprising 70 mass % or more, in total, of the amorphous oxidized glutathione and the water-soluble cellulose derivative based on the total amount of the solid composition.

The present specification encompasses the contents disclosed in JP Patent Application No. 2018-066497 to which the present application claims priority.

The solid composition comprising oxidized glutathione of one or more embodiments of the present invention can be produced by a simple method. The solid composition has low deliquescence and is easy to handle.

### BRIEF DESCRIPTION OF THE DRAWINGS

FIG. 1 shows appearances of spray dried powders prepared in Experiment 2 immediately before the start of test (day 0) and after 48-hour storage (day 2) stored at 25° C. under the atmosphere having a relative humidity of 69%.

FIGS. 2A-2B show the results of agitation granulation using the spray dried powder of one or more embodiments of the present invention as a GSSG starting material powder in Experiment 5. The photograph of FIG. 2A shows the state in the granulator when the agitation granulation is completed. The photograph, left, of FIG. 2B shows particles passing through a sieve having an opening of 2 mm contained in the agitation granulated product using the spray dried powder of one or more embodiments of the present invention, and the photograph, right, of FIG. 2B shows particles remaining on the sieve having an opening of 2 mm contained in the above agitation granulated product.

FIGS. 3A-3B show the results of agitation granulation using the conventional spray dried powder as a GSSG starting material powder in Experiment 5. The photograph of FIG. 3A shows the state in the granulator when the agitation granulation is completed. The photograph, left, of FIG. 3B shows particles passing through a sieve having an opening of 2 mm in the agitation granulated product using the conventional spray dried powder, and the photograph, right, of FIG. 3B shows particles remaining on a sieve having an opening of 2 mm in the above agitation granulated product.

### DETAILED DESCRIPTION OF THE EMBODIMENTS

### <1. Oxidized Glutathione>

Oxidized glutathione (GSSG) is a substance formed when 2 molecules of reduced glutathione (GSH, N-(N-γ-L-glutamyl-L-cysteinyl)glycine) bind to each other via a disulfide bond.

In one or more embodiments of the present invention, the oxidized glutathione (GSSG) can encompass various forms of GSSG such as a free form which is not bound to other substances or ionized, a salt formed by GSSG with an acid or a base, a hydrate thereof, and a mixture thereof. Similarly, in one or more embodiments of the present invention, the reduced glutathione (GSH) can also encompass various forms of GSH such as a free form which is not bound to other substances or ionized, a salt formed by GSH with an acid or a base, a hydrate thereof, and a mixture thereof.

GSSG used in one or more embodiments of the present invention may be a mixture with GSH, an amount of GSSG may be relatively larger than an amount of GSH or the GSSG may comprise substantially no GSH. In one or more embodiments, the total mass (a mass all in terms of a free form) of GSSG based on the total mass (a mass all in terms of a free form) of GSSG and GSH may be 70 mass % or more, 80 mass % or more, 90 mass % or more, 95 mass % or more, 98 mass % or more, or 100 mass %.

The salt of GSSG is not particularly limited. The salt of GSSG may be one or more salts such as an ammonium salt, a calcium salt, a magnesium salt, a sodium salt, and a lithium salt. The salt of GSSG may typically be one or more salts selected from an ammonium salt, a calcium salt and a magnesium salt. Examples of the GSSG salt include a 1 ammonium salt of GSSG, a 0.5 calcium salt of GSSG or 1 calcium salt of GSSG, and a 0.5 magnesium salt of GSSG or 1 magnesium salt of GSSG.

Oxidized glutathione used is not particularly limited and can be, for example, a commercially available product, or those obtained by preparing reduced glutathione by a known method such as a fermentation method and oxidizing the reduced glutathione by a known method.

### <2. Water-Soluble Cellulose Derivative>

The present inventors surprisingly found that a solid composition comprising an amorphous oxidized glutathione obtained by drying a solution comprising oxidized glutathione and a water-soluble cellulose derivative dissolved in an aqueous solvent has low deliquescence and is easy to handle. When a polymer compound other than a water-soluble cellulose derivative is used, such an effect cannot be obtained. Patent Literature 4 describes a controlled-release fertilizer comprising oxidized glutathione and sodium carboxymethyl cellulose or a salt thereof, but crystals are used as the oxidized glutathione which is different from the solid composition of one or more embodiments of the present invention.

Examples of the water-soluble cellulose derivative include, specifically, carboxymethyl cellulose, hydroxypropylmethyl cellulose, hydroxyethyl cellulose, hydroxypropyl cellulose, methyl cellulose and ethyl cellulose and salts thereof, and one of these can be used singly or in combination of two or more. Examples of the salt include a sodium salt, a potassium salt and a lithium salt. Particularly, one or more selected from carboxymethyl cellulose and hydroxypropylmethyl cellulose and salts thereof are preferable as the water-soluble cellulose derivative.

The water-soluble cellulose derivative may have 1% viscosity at 25° C. of 10,000 mPa·s or less, 5000 mPa·s or less, 2000 mPa·s or less, or 500 mPa·s or less.

### <3. Solid Composition>

One or more embodiments of the present invention primarily relate to the solid composition comprising an amorphous oxidized glutathione and a water-soluble cellulose derivative.

The solid composition of one or more embodiments of the present invention has low deliquescence in the air and thus is easy to hand while comprising oxidized glutathione. Therefore, the solid composition is preferable. The solid composition of one or more embodiments of the present invention has low deliquescence in the air and thus is easy to mix with other components, which means that the solid composition of one or more embodiments of the present invention is suitable as a GSSG starting material powder. The GSSG starting material powder may be used for applications such as a fertilizer by being blended with other components.

It can be confirmed by a means such as X-ray diffraction that the oxidized glutathione is amorphous.

In the solid composition of one or more embodiments of the present invention, a blending ratio of the amorphous oxidized glutathione to the water-soluble cellulose derivative is not particularly limited and, in one or two or more embodiments, 1 part by mass or more, or 2 parts by mass or more of the water-soluble cellulose derivative per 100 parts by mass of the amorphous oxidized glutathione. A solid composition having a blending ratio of the amorphous oxidized glutathione to the water-soluble cellulose derivative within this range has particularly low deliquescence. The upper limit of an amount of a water-soluble cellulose derivative per 100 parts by mass of the amorphous oxidized glutathione is not particularly limited and, in one or two or more embodiments, 100 parts by mass or less, 50 parts by mass or less, or 10 parts by mass or less of the water-soluble cellulose derivative per 100 parts by mass of the amorphous oxidized glutathione.

The amount of the amorphous oxidized glutathione in the solid composition of one or more embodiments of the present invention is not particularly limited. A higher proportion of the amorphous oxidized glutathione is more preferable as a GSSG starting material powder. The solid composition in one or more embodiments of the present invention may comprise, based on the total amount of the solid composition, 20 mass % or more, 25 mass % or more, 30 mass % or more, 40 mass % or more, 50 mass % or more, 60 mass % or more, 65 mass % or more, 70 mass % or more, 80 mass % or more, or 90 mass % or more, of the amorphous oxidized glutathione.

The total amount of the amorphous oxidized glutathione and the water-soluble cellulose derivative in the solid composition of one or more embodiments of the present invention may be, based on the total amount of the solid composition, 70 mass % or more, 80 mass % or more, 90 mass % or more, 95 mass % or more, or 98 mass % or more.

### <4. Method for Producing Solid Composition>

The method for producing the solid composition of one or more embodiments of the present invention is not particularly limited and, in one or two or more embodiments, it is a method comprising a step of drying a solution comprising oxidized glutathione and a water-soluble cellulose derivative dissolved in an aqueous solvent.

The aqueous solvent here can be a water-based liquid. Examples of the aqueous solvent include water and a mixed solvent of water and a water-soluble solvent. Examples of the water-soluble solvent include alcohols such as ethanol.

An example of the solution comprising oxidized glutathione and a water-soluble cellulose derivative dissolved in an aqueous solvent can include a solution prepared by adding a water-soluble cellulose derivative to an oxidized glutathione solution, wherein the oxidized glutathione solution can be obtained by preparing reduced glutathione by a known method such as a fermentation method and then oxidizing the reduced glutathione by an oxidation reaction in an aqueous solvent, or by isolating oxidized glutathione from the oxidized glutathione solution and as needed further purifying the isolated product, then adding the isolated product or the purified product to an aqueous solvent. The above isolation can include, for example, concentration, dilution and/or filtration of the solution.

The above oxidation reaction proceeds in a suitable aqueous solvent such as water using an oxidizing agent. Examples of the oxidizing agent include weak oxidizing agents such as oxygen; and strong oxidizing agents such as hydrogen peroxide, iodine and potassium ferrocyanide. Further, other types of oxidizing agents, such as gaseous substance (for example, gaseous nitrogen oxide) and sulfoxide, may also be used. In this oxidation reaction, for example, an oxidation catalyst such as copper sulfate, iron sulfate, iron (III) chloride can be used as needed. Additionally, the above oxidation reaction may include controlling pH of the reaction solution. The pH can be controlled to be, for example, 5 to 12, 6 to 10, or 7 to 9. When pH is adjusted to be within the above range, oxidized glutathione can be stabilized and a reaction rate can be increased.

The concentrations of the oxidized glutathione and the water-soluble cellulose derivative in the solution comprising the oxidized glutathione and the water-soluble cellulose derivative dissolved in an aqueous solvent are not particularly limited and can be the concentrations at which the solvent can be removed in the step of drying the solution. The amount and ratio of the oxidized glutathione and the water-soluble cellulose derivative in the solution can be determined in accordance with the amount and ratio of each component in a solid composition to be obtained. In one or more embodiments, an amount and/or a ratio of the oxidized glutathione and the water-soluble cellulose derivative in the components (solid matters which do not volatilize when dried) other than the solvent in the solution is set in such a way as to be in a preferable range described as the amount and/or the ratio of each component in the solid composition.

The step of drying the solution can be carried out by a drying method such as spray drying, freeze drying or drum drying.

### EXAMPLES

### <Experiment 1>

Aqueous solutions containing 17 mass % of oxidized glutathione (GSSG) and any one of the excipients shown in the following table in an amount of 10 parts by mass per 100 parts by mass of GSSG (parts by mass in terms of the mass of a free form; the same shall apply hereinafter) were prepared. For a control experiment, an aqueous solution containing 17 mass % of GSSG but not containing an excipient was prepared.

The aqueous solution was spray dried to obtain a spray dried powder.

The spray drying was carried out using Mini Spray Dryer B-290 manufactured by Nihon BUCHI K.K. under conditions of an inlet temperature of 170° C. and an outlet temperature of 90 to 110° C. (the outlet temperature was not controlled).

The spray dried powder was allowed to stand for 24 hours at 40° C. under the atmosphere having a relative humidity of 75%. The spray dried powder was visually observed to confirm the presence or absence of deliquescence.

TABLE 1

| | | | Presence of |
|---|---|---|---|
| | Excipient | Product name | deliquescence |
| Control | None | — | Yes |
| Example 11 | Sodium carboxymethyl | Celogen ™6A | No |
| | cellulose (CMC Na) | (DKS Co. Ltd.) | |
| Example 12 | CMC Na | Celogen ™ WS-C | No |
| | | (DKS Co. Ltd.) | |
| Example 13 | CMC Na | Celogen ™ PR | No |
| | | (DKS Co. Ltd.) | |
| Example 14 | CMC Na | Celogen ™ 3H | No |
| | | (DKS Co. Ltd.) | |
| Example 15 | CMC Na | Celogen ™ BSH-12 | No |
| | | (DKS Co. Ltd.) | |
| Example 16 | CMC Na | Celogen ™ HE-1500F | No |
| | | (DKS Co. Ltd.) | |
| Example 17 | CMC Na | Celogen ™ F-AG | No |
| | | (DKS Co. Ltd.) | |
| Example 18 | Hydroxypropylmethyl | METOLOSE ™ | No |
| | cellulose (HPMC) | SE-06 (Shin-Etsu | |
| | | Chemical Co., Ltd.) | |
| Comparative | Polyvinyl alcohol | Denka HV polymer ™ | Yes |
| Example 11 | (PVA) | F-300S (Denka | |
| | | Company Limited) | |
| Comparative | Pregelatinized | Corn alpha Y | Yes |
| Example 12 | starch | (Sanwa Starch | |
| | | Co., Ltd.) | |
| Comparative | Pregelatinized | Industrial tapioca | Yes |
| Example 13 | starch | alpha (Sanwa Starch | |
| | | Co., Ltd.) | |
| Comparative | Pregelatinized | Matsunorin ™ M | Yes |
| Example 14 | starch | (Matsutani Chemical | |
| | | Industry Co., Ltd.) | |
| Comparative | Oxidized starch | Matsunorin ™ GS | Yes |
| Example 15 | | (Matsutani Chemical | |
| | | Industry Co., Ltd.) | |
| Comparative | Dextrin | Max 1000 | Yes |
| Example 16 | | (Matsutani Chemical | |
| | | Industry Co., Ltd.) | |
| Comparative | Dextrin | Pinedex ™ #1 | Yes |
| Example 17 | | (Matsutani Chemical | |
| | | Industry Co., Ltd.) | |
| Comparative | Dextrin | Hidex 128 | Yes |
| Example 18 | | (JAPAN CORN | |
| | | STARCH CO., LTD.) | |
| Comparative | Trehalose | TREHA | Yes |
| Example 19 | | (Hayashibara | |
| | | Co., Ltd.) | |

### <Experiment 2>

Aqueous solutions containing 11 mass % of oxidized glutathione (GSSG) and any one of the excipients shown in the following table in an amount of 10 parts by mass or 3 parts by mass per 100 parts by mass of GSSG were prepared. For a control experiment, an aqueous solution containing 11 mass % of GSSG but not containing an excipient was prepared.

The aqueous solution was spray dried to obtain a spray dried powder.

The spray drying was carried out using Mobile Miner 2000 manufactured by Nitro Corporation under conditions of an inlet temperature of 170° C. and an outlet temperature of 85 to 90° C. (the outlet temperature was not controlled).

The spray dried powder was allowed to stand for 48 hours at 25° C. under the atmosphere having a relative humidity of 69%. The spray dried powder was visually observed to confirm the presence or absence of deliquescence.

TABLE 2

| | | | Presence of |
|---|---|---|---|
| | Excipient | Product name | deliquescence |
| Control | None | — | Yes |
| Example 21 | 10 Parts by mass (per 100 parts | Celogen ™ 6A | No |
| | by mass of GSSG) of CMC Na | (DKS Co. Ltd.) | |
| Example 22 | 10 Parts by mass (per 100 parts | Celogen ™ WS-C | No |
| | by mass of GSSG) of CMC Na | (DKS Co. Ltd.) | |
| Example 23 | 3 Parts by mass (per 100 parts | METOLOSE ™ SE-06 | No |
| | by mass of GSSG) of HPMC | (Shin-Etsu Chemical Co., Ltd.) | |

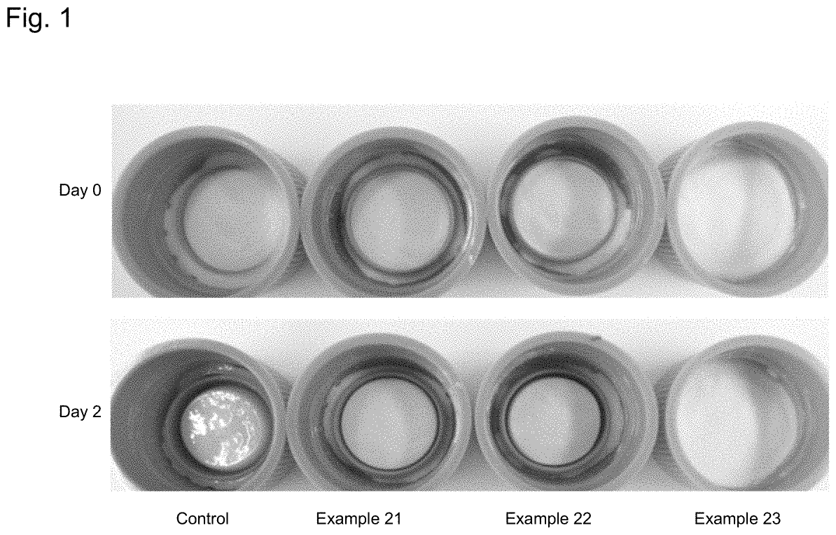

FIG. 1 shows appearances of each spray dried powders immediately before the start of storage test (day 0) and after 48-hour storage (day 2) under the atmosphere described above.

### <Experiment 3>

Aqueous solutions containing 11 mass % of oxidized glutathione (GSSG) and Celogen™ WS-C (DKS Co. Ltd.) (CMC Na) in an amount of 1 part by mass, 2 parts by mass, 3 parts by mass, 5 parts by mass, 10 parts by mass or 50 parts by mass, per 100 parts by mass of GSSG were prepared.

The aqueous solution was spray dried to obtain a spray dried powder.

The spray drying was carried out using Mini Spray Dryer B-290 manufactured by Nihon BUCHI K.K. under conditions of an inlet temperature of 170° C. and an outlet temperature of 90 to 110° C. (the outlet temperature was not controlled).

The spray dried powder was allowed to stand for 24 hours at 40° C. under the atmosphere having a relative humidity of 75%. The spray dried powder was visually observed to confirm the presence or absence of deliquescence.

TABLE 3

| | | Presence of |
|---|---|---|
| | Amount of CMC Na added | deliquescence |
| Example 31 | 1 Part by mass (per 100 parts | No |
| | by mass of GSSG) of CMC Na | |
| Example 32 | 2 Parts by mass (per 100 parts | No |
| | by mass of GSSG) of CMC Na | |
| Example 33 | 3 Parts by mass (per 100 parts | No |
| | by mass of GSSG) of CMC Na | |
| Example 34 | 5 Parts by mass (per 100 parts | No |
| | by mass of GSSG) of CMC Na | |
| Example 35 | 10 Parts by mass (per 100 parts | No |
| | by mass of GSSG) of CMC Na | |
| Example 36 | 50 Parts by mass (per 100 parts | No |
| | by mass of GSSG) of CMC Na | |

### <Experiment 4>

Aqueous solutions containing 11 mass % of oxidized glutathione (GSSG) and Celogen™ WS-C (DKS Co. Ltd.) (CMC Na) in an amount of 3 parts by mass or 10 parts by mass per 100 parts by mass of GSSG were prepared. For a control experiment, an aqueous solution containing 11 mass % of GSSG but not containing an excipient was prepared.

The aqueous solutions were dried by freeze drying or drum drying to obtain dried powders.

The freeze drying was carried out using Freeze Dryer FD-5N manufactured by TOKYO RIKAKIKAI CO., LTD.

The drum drying was carried out using a drum dryer manufactured by KATSURAGI INDUSTRY CO., LTD. (drum size: φ400 mm×L500, drum surface area: 0.625 m²×2 drums, drum model system: double, supplying width: 500 mm, drum effective area: 1.25 m²) under the conditions of vapor pressure: 0.3 MPa, drum rotation rate 0.2 rpm and drum gap less than 0.1 mm.

The dried powder was allowed to stand for 24 hours at 40° C. under the atmosphere having a relative humidity of 75%. The dried powder was visually observed to confirm the presence or absence of deliquescence.

TABLE 4

| | | | Presence of |
|---|---|---|---|
| | Amount of CMC Na added | Drying method | deliquescence |
| Control 41 | — | Freeze drying | Yes |
| Example 41 | 3 Parts by mass (per 100 parts by mass of GSSG) of CMC Na | Freeze drying | No |
| Example 42 | 10 Parts by mass (per 100 parts by mass of GSSG) of CMC Na | Freeze drying | No |
| Control 42 | — | Drum drying | Yes |
| Example 43 | 10 Parts by mass (per 100 parts by mass of GSSG) of CMC Na | Drum drying | No |

### <Experiment 5>

Granulation by agitation granulation was carried out using, as a GS SG starting material powder, the spray dried powder (the spray dried powder of one or more embodiments of the present invention) prepared in Example 21 of Experiment 2 or the spray dried powder (conventional spray dried powder) prepared in the control experiment of Experiment 2 in combination with other components.

Granulation method: 17.1 mass % of the GSSG starting material powder, 35.6 mass % of ammonium sulfate (manufactured by Sumitomo Chemical Co., Ltd.), 20.0 mass % of monobasic ammonium phosphate (manufactured by Shimonoseki Mitsui Chemicals, Inc.), 24.3 mass % of potassium sulfate (manufactured by MITSUI & CO., LTD.), 1 mass % of linear alkylbenzene sulfonate (manufactured by Lion Corporation), and 2 mass % of sodium carboxymethyl cellulose (manufactured by DKS Co. Ltd.) were mixed and an appropriate amount of water (4 parts by mass) per 100 parts by mass of the mixture was added to the mixture. The resultant mixture was granulated by using Sample Mill manufactured by Kyoritsu Riko Co., Ltd.

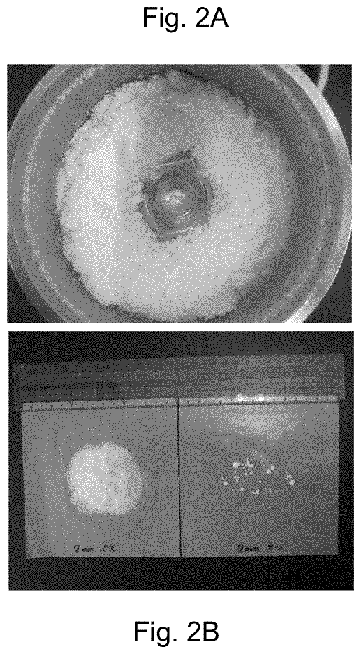

The results of agitation granulation using the spray dried powder of one or more embodiments of the present invention as the GSSG starting material powder are shown in FIGS. 2A-2B. The photograph of FIG. 2A shows the state in the granulator when the agitation granulation is completed. The photograph, left, of FIG. 2B shows particles passing through a sieve having an opening of 2 mm contained in the agitation granulated product using the spray dried powder of one or more embodiments of the present invention, and the photograph, right, of FIG. 2B shows particles remaining on a sieve having an opening of 2 mm contained in the above agitation granulated product. Most particles contained in the granulated product were fine particles passing through a sieve having an opening of 2 mm.

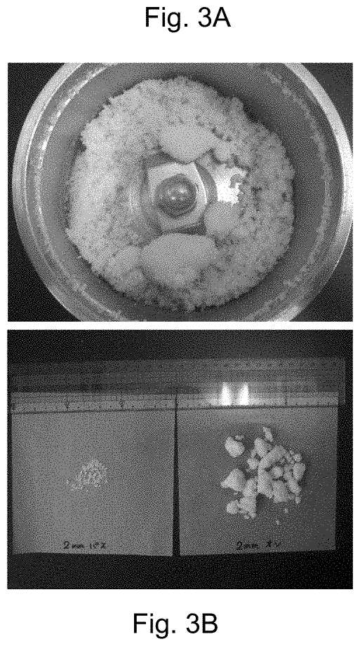

FIGS. 3A-3B shows the results of agitation granulation using the conventional spray dried powder as the GSSG starting material powder. The photograph of FIG. 3A shows the state in the granulator when the agitation granulation is completed. The photograph, left, of FIG. 3B shows particles passing through a sieve having an opening of 2 mm contained in the agitation granulated product using the conventional spray dried powder. The photograph, right, of FIG. 3B shows particles remaining on a sieve having an opening of 2 mm contained in the above agitation granulated product. Most particles contained in the granulated product were masses in the size incapable of passing through a sieve having an opening of 2 mm.

### <Experiment 6>

The GSSG-containing dried powders of Examples 11 to 18 of Experiment 1, Examples 21 to 23 of Experiment 2, Examples 31 to 36 of Experiment 3, and Examples 41 to 43 of Experiment 4 were used as samples and confirmed for X-ray diffraction images using a powder X-ray diffractometer (Mini Flex II manufactured by RIGAKU Corporation) whereby no characteristic peaks in crystals were detected. These findings confirmed that each of the powders contained amorphous GSSG.

All publications, patents and patent applications cited in the present specification are incorporated herein by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A method for producing a solid composition comprising an amorphous oxidized glutathione and a water-soluble cellulose derivative, the method comprising:
drying a solution comprising an oxidized glutathione and the water-soluble cellulose derivative dissolved in an aqueous solvent.

2. The method according to claim 1, wherein said drying comprises drying the solution by spray drying, freeze drying or drum drying.

3. The method according to claim 1, wherein the solution comprises 1 to 50 parts by mass of the water-soluble cellulose derivative per 100 parts by mass of the oxidized glutathione.

4. The method according to claim 1, wherein the solution comprises 20 mass % or more of the oxidized glutathione based on a total amount of components other than a solvent.

5. The method according to claim 1, wherein the solution comprises 70 mass % or more, in total, of the oxidized glutathione and the water-soluble cellulose derivative based on a total amount of components other than a solvent.

6. A solid composition comprising:
an amorphous oxidized glutathione; and
a water-soluble cellulose derivative.

7. The solid composition according to claim 6, comprising 1 to 50 parts by mass of the water-soluble cellulose derivative per 100 parts by mass of the amorphous oxidized glutathione.

8. The solid composition according to claim 6, comprising 20 mass % or more of the amorphous oxidized glutathione based on a total amount of the solid composition.

9. The solid composition according to claim 6, wherein the solid composition is produced by a method comprising:
drying a solution comprising an oxidized glutathione and the water-soluble cellulose derivative dissolved in an aqueous solvent.

10. The solid composition according to claim 9, wherein said drying comprises drying the solution by spray drying, freeze drying or drum drying.

11. The solid composition according to claim 6, comprising 70 mass % or more, in total, of the amorphous oxidized glutathione and the water-soluble cellulose derivative based on a total amount of the solid composition.
